SEQUENCE LISTING (6 sequences)

SEQ ID NO: 1 - DNA, 51 bp, Artificial Sequence
  Synthetic Construct
ctagactcgagtagcggccgcggcagcgactacaaggacgacgacgacaag

SEQ ID NO: 2 - protein, 45 aa, Artificial Sequence
  Synthetic Construct
GLYSERASPTYRLYSASPASPASPASPLYSGLYSERCYSAREALA

SEQ ID NO: 3 - DNA, 61 bp, Artificial Sequence
  Synthetic Construct
ctagactcgagtagcggccgcggaggcggaggaagcggaggaggagggagctgccgcgcgc

SEQ ID NO: 4 - protein, 39 aa, Artificial Sequence
  Synthetic Construct
GLYGLYGLYGLYSERGLYGLYGLYGLYSERCYSARGALA

SEQ ID NO: 5 - DNA, 1369 bp, Artificial Sequence
  Synthetic Construct
tctagactcgagtagcggccgcggcagcgactacaaggacgacgacgacaaggggagctgccgcgcgctggaggtggcaaatggcacagtgacaagcctgccaggggccaccgttaccctgatttgccccgggaaggaagcagcaggcaatgttaccattcactgggtgtactctggctcacaaaacagagaatggactaccacaggaaacacactggttctgagggacgtgcagctcagcgacactggggactatttatgctccctgaatgatcacctggtggggactgtgcccttgctggtggatgttcccccagaggagcccaagctctcctgcttccggaagaacccccttgtcaacgccatctgtgagtggcgtccgagcagcaccccctctccaaccacgaaggctgtgctgtttgcaaagaaaatcaacaccaccaacgggaagagtgacttccaggtgccctgccagtattctcagcagctgaaaagcttctcctgccaggtggagatcctggagggtgacaaagtataccacatagtgtcactgtgcgttgcaaacagtgtgggaagcaagtccagccacaacgaagcgtttcacagcttaaaaatggtgcagccggatccacctgccaaccttgtggtatcagccatacctggaaggccgcgctggctcaaagtcagctggcagcaccctgagacctgggacccgagttactacttgctgcagttccagcttcgataccgacctgtatggtcaaaggagttcacggtgttgctgctcccggtggcccagtaccaatgcgtcatccatgatgccttgcgaggagtgaagcacgtggtccaggtccgtgggaaggaggagcttgaccttggccagtggagtgaatggtccccagaggtcacgggcactccttggatagcagagcccaggaccaccccggcaggaatcctctggaaccccacacaggtctctgttgaagactctgccaaccacgaggatcagtacgaaagttctacagaagcaacgagtgtcctcgccccagtgcaagaatcctcgtccatgtccctgcccacattcctggtagctggaggaagcttggcgtttgggttgcttctctgtgtcttcatcatcctgagactcaagcagaaatggaagtcagaggctgagaaggaaagcaagacgacctctcctccacccccaccgtattccttgggcccactgaagccgaccttccttctggttcctctcctcaccccacacagctctgggtctgacaataccgtaaaccacagctgcctgggtgtcagggacgcacagagcccttatgacaacagcaacagagactacttattccccagataa

SEQ ID NO: 6 - DNA, 1363 bp, Artificial Sequence
  Synthetic Construct
tctagactcgagtagcggccgcggaggcggaggaagcggaggaggagggagctgccgcgcgctggaggtggcaaatggcacagtgacaagcctgccaggggccaccgttaccctgatttgccccgggaaggaagcagcaggcaatgttaccattcactgggtgtactctggctcacaaaacagagaatggactaccacaggaaacacactggttctgagggacgtgcagctcagcgacactggggactatttatgctccctgaatgatcacctggtggggactgtgcccttgctggtggatgttcccccagaggagcccaagctctcctgcttccggaagaacccccttgtcaacgccatctgtgagtggcgtccgagcagcaccccctctccaaccacgaaggctgtgctgtttgcaaagaaaatcaacaccaccaacgggaagagtgacttccaggtgccctgccagtattctcagcagctgaaaagcttctcctgccaggtggagatcctggagggtgacaaagtataccacatagtgtcactgtgcgttgcaaacagtgtgggaagcaagtccagccacaacgaagcgtttcacagcttaaaaatggtgcagccggatccacctgccaaccttgtggtatcagccatacctggaaggccgcgctggctcaaagtcagctggcagcaccctgagacctgggacccgagttactacttgctgcagttccagcttcgataccgacctgtatggtcaaaggagttcacggtgttgctgctcccggtggcccagtaccaatgcgtcatccatgatgccttgcgaggagtgaagcacgtggtccaggtccgtgggaaggaggagcttgaccttggccagtggagtgaatggtccccagaggtcacgggcactccttggatagcagagcccaggaccaccccggcaggaatcctctggaaccccacacaggtctctgttgaagactctgccaaccacgaggatcagtacgaaagttctacagaagcaacgagtgtcctcgccccagtgcaagaatcctcgtccatgtccctgcccacattcctggtagctggaggaagcttggcgtttgggttgcttctctgtgtcttcatcatcctgagactcaagcagaaatggaagtcagaggctgagaaggaaagcaagacgacctctcctccacccccaccgtattccttgggcccactgaagccgaccttccttctggttcctctcctcaccccacacagctctgggtctgacaataccgtaaaccacagctgcctgggtgtcagggacgcacagagcccttatgacaacagcaacagagactacttattccccagataa

(19) United States
(12) Patent Application Publication
(10) Pub. No.: US 2006/0115807 A1
(43) Pub. Date: Jun. 1, 2006

(54) Episomal expression system

(76) Inventor: Ian Paul Chambers, Edinburgh (GB)

Correspondence Address: TUCKER, ELLIS & WEST LLP, 1150 HUNTINGTON BUILDING, 925 EUCLID AVENUE, CLEVELAND, OH, 44115-1414, US

(21) Appl. No.: 10/827,684

(22) Filed: Apr. 19, 2004

Related U.S. Application Data
(63) Continuation-in-part of application No. 09/359,672, filed on Jul. 23, 1999, now abandoned.

(30) Foreign Application Priority Data
Jan. 24, 1997 (GB) GB 9701492.2
Jan. 26, 1998 (WO) PCT/GB98/00216
Mar. 29, 2004 (GB) GB 0407043.9

(51) Int. Cl. C12Q 1/70; C12Q 1/68; C12N 5/06; C12N 15/86

(52) U.S. Cl. 435/5; 435/6; 435/456; 435/349; 435/354

(57) ABSTRACT

A cell which expresses a replication factor is transfected with a vector which requires the presence of the replication factor to be maintained episomally within the cell. This is then expanded into a plurality of cells; and a cell in the plurality of cells selected (i) which maintains the vector episomally, and (ii) in which the vector has not integrated into chromosomal DNA of the cell.

### FIELD OF INVENTION

This invention relates to methods of expressing DNA episomally in cells, to vectors for expression of DNA in cells and to transfected cells. The invention also relates to assays carried out in transfected cells or differentiated derivatives of such cells. In particular the invention relates to transfection of and expression of DNA in embryonic and more particularly embryonic stem (ES) cells.

### BACKGROUND TO INVENTION

The wealth of sequence information now becoming available from the genome projects demands the development of new, high throughput systems for functional analysis. A powerful route to discovering and characterising genes involved in determination and differentiation in mammals is potentially available via the genetic manipulation of ES cells in vitro.

ES cells, which are derived from the pluripotential inner cell mass (ICM) of the preimplantation mouse embryo (2,3), retain the capacity for multilineage differentiation both in vitro (4,5) and in vivo (6,7). In principle, therefore, gene products which influence developmental decisions should be assayable in ES cell culture systems, whatever the source of the cells. However, there are major difficulties in analysing cDNA function by ES cell transfection. The frequency of isolating stable transfectants is low (<10⁻⁴ by electroporation, calcium phosphate co-precipitation or lipofection) and the great majority of transfectants show heterogeneous and unstable expression.

These problems are particularly significant in the case of cDNAs whose expression causes differentiation because differentiated ES cell progeny do not generally proliferate. In such cases transfectants may still be isolated but transgene expression will be minimal.

Episomal vectors have been used for functional screening in other cell types in order to increase the frequency of stable transfection and to achieve reliable transgene expression. However, previously described episomal vectors, for example based on Epstein-Barr virus (EBV) or bovine papilloma virus (BPV), have limitations both in host cell range and maintenance during long-term culture.

A modified extrachromosomal vector is known based on the replication system of murine polyoma virus (8). This plasmid, pMGD20neo, can be stably maintained as an episome in ES cells during long term culture, and these cells can be further transfected with a second episomal vector, albeit at efficiences of less than 1%. Importantly, the low levels of large T protein produced have no overt effect on the growth or differentiation properties of the ES cells (8,9). It is also known to use pMGD20neo for cDNA expression. However, this vector already comprises two expression cassettes, one each for large T antigen and the neo selectable marker so its size constrains its use for expression of a third cassette containing a cDNA.

It is an object of the invention to provide a vector for transfection of and expression of DNA within a cell and a method of expressing DNA in a cell that overcomes or at least ameliorates the disadvantages identified in the art. An object of at least the preferred embodiments of the invention is to achieve, in a transfected cell, expression that is more stable and more homogenous than hitherto attainable. Further objects of preferred embodiments of the invention are to provide a method of expressing a DNA in an embryonic cell in a more stable and more homogenous manner than hitherto attainable, and to provide for stable transfection of embryonic cells at a higher frequency than can be obtained using conventional vectors.

### SUMMARY OF INVENTION AND DESCRIPTION OF PREFERRED EMBODIMENTS

The invention is based upon the maintenance of a vector within a cell, wherein maintenance of the vector is dependant upon the continued presence within the cell of a certain factor and wherein that factor is not expressed by the vector but is produced in or present in the cell in an amount sufficient to maintain the vector.

Accordingly the invention provides a transfection and expression method comprising, in a cell that expresses or will express a replication factor, introducing a vector dependent upon that replication factor. First aspects of the invention provide an expression method and system and vectors therefor. Additional aspects provide an improvement thereon, including cells which can be transfected and used in screening assays.

Thus, in a first aspect, the invention provides a method of expressing a DNA in a cell, comprising:
(a) (i) transfecting the cell with a first vector that expresses a replication factor; or
   (ii) otherwise obtaining a cell that expresses or will express the replication factor; and
(b) transfecting the cell with a second vector, wherein
   (i) the second vector contains a DNA, or is adapted to receive a DNA, in operative combination with a promoter for expression of the DNA; and
   (ii) extrachromosomal replication of the second vector is dependant upon presence within the cell of the replication factor.

The replication factor is optionally non-toxic to the cell. Alternatively, the replication factor is toxic to the cell at high levels of expression but at low levels of expression is substantially non-toxic to the cell but at these low levels is present in sufficient amount to enable replication of the second vector.

Further, the replication factor preferably does not alter the ability of the cell to differentiate or proliferate, and may thus be regarded as being neutral to the cell phenotype. This enables the activities of the product of a cDNA to be investigated over a long time period and many cell generations without having to take account of possible interfering effects of the replication factor present within the cell. Again, the replication factor may be phenotype-neutral at all levels or may be neutral at a low level which is nevertheless a sufficient level to maintain the second vector within the cell.

The invention is of application to all cell types for which there exists, whether from a natural or synthetic source, a replication factor capable of maintaining in that cell type an episomal vector. The vector is preferably stably maintained, meaning it is maintained over a number of cell generations, and at least over 3 generations. The cell is preferably selected from the group consisting of mammalian cells, in particular primate cells or murine cells, and avian cells, especially rodent such as mouse and rat cells and human cells. It is further preferred that the cell is pluripotent, especially an embryonic cell, in particular an ES, EC (embryonic carcinoma) or EG (embryonic gonadal) cell, or differentiated progeny of any such cell.

While reference is made to the second vector, it will be appreciated that the replication factor is optionally present in the cell other than following transfection with a first vector. For example a culture of cells that already express the replication factor may be obtainable from a third party.

In an embodiment of the invention described in detail below, the method comprises transfecting an ES cell with a first vector that expresses a viral replication factor, and thereafter transfecting the ES cell with a second vector that expresses a cDNA and is dependant upon presence of the viral replication factor for its extrachromosomal replication within the ES cell. The frequency of the first transfection step is generally low and may result in as few as 1 in 10⁵ successful stable transfectants—this level of success is recognised as typical in this art. However, the second transfection has surprisingly and advantageously found to result in a significantly higher frequency of successful stable transfectant colonies being obtained. The second transfection can be carried out with approximately a 1% or higher success rate.

One suitable viral replication factor for mouse cells, in particular mouse ES cells, is polyoma large T antigen, in which case the cell of step (a) expresses the polyoma large T antigen and the second vector comprises an origin of replication that binds the polyoma large T antigen, such as the polyoma replication origin, referred to as Ori. Another suitable viral replication factor for primate cells is based upon Epstein Barr virus, in which the primate cell of step (a) expresses the EBNA-1 antigen and the second vector comprises an origin of replication that binds EBNA-1, such as OriP. Viral replication factors are generally species—specific and so expression of DNA according to the invention is dependent upon choice of a replication factor appropriate to the cell. Polyoma large T has been described for use in mouse cells. EβNA-1 is suitable for human cells. Still further systems are optionally based on papilloma virus replication factors, for human cells, or SV40 virus large T antigen, for simian cells, and further suitable replication factors may also be selected from functional variants, derivatives and analogues of these replication factors, such as temperature sensitive variants.

In use, the second vector is constructed according to standard techniques so as to contain a cDNA sequence or insert of interest operatively combined with a promoter to express the cDNA. The second vector is used to transfect an ES cell already expressing a replication factor and successful transfectants are recovered in which it is found that the second vector is stably maintained within the ES cell and expresses the cDNA with a more homogenous pattern than when prior art techniques are followed. Thus, the invention provides an advantageous method for expression of a cDNA in a cell.

In this context, “homogenous” in relation to expression of a cDNA in a colony of transfected ES cells is used to indicate that most cells, or a large proportion of cells, or preferably most cells, or more preferably substantially all cells, express the cDNA and “stable” is used to indicate that the cells continue to express the cDNA at a similar level and preferably at substantially the same level. In the examples carried out to date and described below, homogenous transfection is seen with the method of the invention to a greater extent than in the art methods. Also, in the examples carried out to date and described below the method results in more stable expression, meaning that expression alters less over time. This has the advantage that study of the effects of a cDNA product over the course of an assay is facilitated.

It is optional for the cell of step (a) first to be obtained or prepared by transfection of a cell by a first vector and for this then to be used for the starting cells for carrying out a plurality of separate transfections by second vectors containing different DNA inserts coding for different DNA products of interest. Following this procedure, the first transfection may be carried out with the level of success typically seen in conventional techniques and the ES cells obtained divided into separate colonies. The second transfections, using different second vectors each with different DNAs of interest are then carried out with the higher levels of success typically seen for the second transfection.

In the case that the method comprises transfection with first and second vectors, it is preferable for the first vector to code for a selectable marker and for the second vector also to code for a selectable marker, though a different one. In a specific embodiment of the invention described below, the first vector codes for hygromycin resistance and the second codes for neomycin resistance. This allows selection of ES cells in which transfection by both first and second vectors has been successful.

It is a further embodiment of the invention for the method to comprise an additional transfection step with a third vector, wherein the third vector contains a cDNA, or is adapted to receive a cDNA, in operative combination with a promoter for expression of the cDNA, and extrachromosomal replication of the third vector is dependant upon presence within the ES cell of the replication factor. Transfection with the third vector is optionally at the same time as transfection with the second vector or subsequent thereto.

The second and third vectors preferably each comprise a selectable marker enabling selection of ES cells in which transfection has been successful. The respective selectable markers are preferably different if the method comprises transfection with both second and third vectors, and preferably different again from the selectable marker of the first vector.

It is a feature of particular embodiments of the invention that the second vector (and third or subsequent vectors if present) are not able to express a functional replication factor. In fact, in construction of the second vector from a vector comprising DNA encoding the replication factor it is preferable for that DNA to be largely or substantially completely deleted.

In a specific embodiment of the invention, the first vector is pMDG20neo and expresses polyoma large T antigen and the second vector comprises the natural target for polyoma large T antigen, namely Ori, expresses a cDNA of interest but does not express large T antigen. In use, the large T antigen is expressed by the first vector and binds to Ori of the second vector when it enters an ES cell, thus enabling replication of the second vector and its maintenance within the ES cell in an extrachromosomal state. In successful transfectants, the vector remains extrachromosomal, and this is believed to render the vector relatively immune from effects seen when a vector is integrated into the host ES cell genome, which effect may include silencing of the cDNA resulting in unstable and heterogeneous expression.

An alternative to use of the first episomal vector is to introduce into the cell a construct that expresses the replication factor and integrates with the cell genome. The construct should therefore include a DNA sequence coding for the replication factor and means for selection of cells in which the construct has successfully integrated; one example is a construct that comprises cDNA coding for, in order, large T antigen—an internal ribosome entry site (IRES)—Bgeo. A culture of cells is then obtained by selecting for cells that express the selectable marker, such as in this case by selection in G418. Staining with Xgal is used to identify transfectant clones which show stable and homogenous expression. The construct preferably comprises a promoter that gives stable, low level expression in transfected cells, such as the HMGCoA promoter for ES cells. The cells obtained can then be subjected to transfection with the second and optionally third and subsequent vectors.

In another embodiment of the invention the second vector comprises an inducible promoter. Some types of differentiated cells, derived from ES cells, can only be obtained with any reliability if a particular differentiating factor is expressed after a prior event. One example is neurone formation which generally only occurs after aggregation of cells. Thus, using an inducible promoter, expression of DNA that codes for the factor that leads to neurone formation can be controlled until the ES cells have suitably aggregated. Interferon responsive promoters are some examples of inducible promoters. Alternatively, the cDNA is designed to be in a non-functional form and to be capable of being modified into a functional form at a later time. One possibility is for the cDNA to be disrupted for example by termination sequences which are flanked by target sites for a site specific recombinase, such as loxP sites, removable by Cre recombinase, or frt sites removable by Flp recombinase. Cre and Flp can be fused to steroid hormone receptors in order to make their activity regulatable. After administration of steroid the Cre or Flp recombinase will translocate to the nucleus and there convert the cDNA into a functional form by excision of the disrupting sequence. It may also be desired to stop or inhibit or reduce replication of the second vector; the method optionally comprises using a site specific recombinase to present replication of the second vector. This can be achieved by deletion of a sequence in the vector to which the replication factor must bind in order for the vector to be replicated by the host cell.

The term DNA or cDNA is usually understood to refer to a DNA sequence that is transcribed into a mRNA that is translated into a polypeptide or protein. In the present invention the term is also intended to encompass any product of DNA expression. It thus includes DNA coding for an antisense RNA, or for an antisense ribozyme molecule.

The method of the invention is suitable for assaying effects of DNA expression, due to the stability and efficiency of expression achievable. Accordingly, the invention further relates to an assay for the effect of presence in a cell of any product of DNA expression—such as protein, polypeptide, antisense RNA, ribozyme RNA, transfer RNA or other. The method comprises steps (a) and (b) as described above wherein the second vector also contains a DNA coding for a selectable marker. The method further comprises selecting for cells that have been transfected with the second vector and maintaining the selected cells over a plurality of generations.

Step (a) may be carried out once and then steps (b) onwards repeated for different assays, and the method is of particular application to screening a cDNA library. Furthermore, two or more cDNAs can be expressed in the same cell to assay the effect of the combination of their respective expression products.

The invention also relates to a vector. Accordingly, the invention provides, in a second aspect, a vector for transfection of an ES cell, wherein:
(i) the vector contains a DNA, or is adapted to receive a DNA, in operative combination with a promoter for expression of the DNA;
(ii) extrachromosomal replication of the vector is dependant upon presence within the ES cell of a replication factor; and
(iii) the vector does not express the replication factor.

The vector is characterized in preferred embodiments as described above in relation to the second vector of the first aspect of the invention.

It is an advantage of at least preferred embodiments of the invention that due to very high efficiency of stable secondary transfection (supertransfection) of cells, for example transfection of ES cells harbouring pMGD20neo with a second plasmid containing the polyoma replication origin (Ori) (8), that expression of DNA is stably and efficiently achieved from the second plasmid.

Another aspect of the present invention provides a method of screening for new DNAs that encode signal sequences and proteins that are transported to the cell surface. The invention accordingly provides a method of investigating the properties of a DNA sequence comprising expressing in a cell a composite DNA including (a) the DNA sequence under investigation, linked to (b) a DNA coding for a cell active protein, wherein
activity of the cell active protein is dependant upon transport of the cell active protein to the cell surface, and
the DNA of (b) does not code for a polypeptide capable of directing transportation of the cell active protein to the cell surface.

This offers the advantage that where the DNA of interest does indeed code for a sequence that transports a polypeptide to the cell surface, whether that polypeptide remains there or is ultimately secreted, this will be apparent from observation that the cell active protein has had or is having its known effect. Thus the method offers a convenient means of identifying DNA sequences that will transport proteins to the cell surface.

The method is suitably used for screening a library of DNAs to identify DNA sequences coding for signal polypeptide sequences that transport proteins to the cell surface. The cell active protein if transported to the cell surface may remain there or be secreted by the cell, and this distinction may be separately assayed, or example by examination of the make-up of the culture medium before and after the investigation.

One convenient way to obtain the DNA of (b) is by deleting or disabling, from a DNA encoding a cell surface or secreted protein, that portion of the DNA that codes for the polypeptide sequence responsible for transportation of the protein to the cell surface. The cell active protein is optionally a cell surface receptor and the DNA of (b) can thus encode a modified form of the receptor preprotein lacking a functional signal sequence. In a specific embodiment described below the IL-6 receptor is used as expression of the receptor in ES cells can be used to inhibit differentiation of the cells—a readily observable property of the cell active protein. Gross morphological or proliferative changes induced in the cell by the cell active protein are of course readily observed, though the invention is of application to any cell active protein whose activity, when it is transported to the cell surface and/or secreted, can be assayed.

A specific embodiment of this aspect of the invention comprises expressing the composite DNA by:
(a) (i) transfecting a cell with a first vector that expresses a replication factor; or
   (ii) otherwise obtaining a cell that expresses the replication factor;
(b) transfecting the cell with a second vector, wherein
   (i) the second vector contains the composite DNA in operative combination with a promoter for expression of the composite DNA;
   (ii) the second vector also contains a DNA coding for a selectable marker in operative combination with a promoter for expression of the selectable marker; and
   (iii) extrachromosomal replication of the second vector is dependant upon presence within the cell of the replication factor;
(c) selecting for cells that have been transfected with the second vector; and
(d) maintaining the selected cells over a plurality of generations so as to assay the effect of expression of the composite DNA.

If many investigations are to be carried out it is preferred that step (a) is carried out once and the cells obtained are divided and used for a plurality of separate methods in which steps (b)-(d) are carried out a plurality of times with second vectors containing different DNA sequences. This offers the advantage that typically the first transfection step is of lower efficiency than the second, so the method avoids having to repeat the low efficiency step too often.

It is particularly preferred that the method is used for identification of a DNA coding for a cell surface or secreted protein, and using the method to screen a library of DNAs provides a means of carrying out the screen for discovery of such DNAs and investigation of their properties. More especially, the method is for discovery of hitherto unknown or uncharacterized cell surface or secreted proteins, or for location of the coding sequence of known proteins of this type.

This aspect of the invention optionally further incorporates in preferred embodiments features of transfection of cells described above in relation to other aspects of the present invention.

The invention enables development of a series of vectors which give highly efficient and robust expression of transgenes in cells. Cloned cDNAs of interest can rapidly be characterised using this system. It is also applicable to the discovery of novel regulatory molecules through functional expression screening of cDNA libraries.

Due to their pluripotent and proliferative character, key cellular processes such as viability, propagation, determination and differentiation, can be analyzed in transfected ES cells. The “supertransfection” system of the invention overcomes the limitations associated with conventional cDNA transfection and opens a powerful new route to gene discovery and characterisation in mammals.

Key features of the episomal supertransfection system, described according to the examples below, are that very high efficiencies of stable transfection are obtained and that cDNA expression is homogeneous, stable and reliably dictated by promoter strength. The increased efficiency of isolating stable transfectants is significant because it allows reliable detection of cDNAs whose expression results in cell death or differentiation. In addition a high transfection efficiency is generally advantageous for any high throughput assay system and is essential for functional cDNA library screening. The reliability of cDNA expression is critical for functional studies and the robust nature of expression from episomal vectors contrasts favourably with the variable and unstable expression observed in conventional ES cell transfectants.

The difference in expression pattern between conventional transfectants and episomal supertransfectants of the invention arises because an extrachromosomal copy of a transgene is not subject to alteration during the integration process nor to modification arising from the genomic sequences flanking an integration site. The so-called “position effect” can modify both the level and pattern of transgene expression in stable transfectants. Furthermore, the expression of integrated transgenes is often suppressed over several generations in ES cell cultures. This silencing phenomenon contributes to the high backgrounds which can be obtained in double replacement type targeting strategies (26). It has been observed in stable transfectants with different transgenes driven by viral promoters or minimal mammalian promoters such as the widely used human β-actin and mouse PGK-1 promoter elements. One hypothesis to explain this phenomenon is that transgenes may become targets of de novo-methyltransferase in stem cells (27). Macleod et al. (28) reported that a methylation free locus could be generated in transgenic mice by introduction of the whole CpG island of the aprt promoter.

Whatever the molecular mechanism of silencing, it appears not to occur to episomally maintained transgenes in vectors of the invention. In addition, the level of expression obtained from vectors of the invention is reliably dictated by promoter strength and can predictably be varied over at least a 10-fold range by appropriate choice of promoter. Episomal constructs of the invention thus offer considerable advantages for functional expression studies in ES cells.

Functional cDNA expression cloning is a powerful method for direct isolation of important genes. The expression screening approach has often been employed to isolate cDNAs encoding surface and secreted molecules via transient expression, for example in COS cells. In a few cases EBV-based systems have also been applied to isolate intracellular regulatory genes via stable expression in the target cells (29-32). The high efficiency of supertransfection in the polyoma system of the invention indicates that this approach could be applied to functional cloning in ES cells. For an effective library screen, the majority of transfectants should only take up a single plasmid. It is also advantageous if the cDNAs can readily be recovered in unrearranged form. Both of these conditions are satisfied by the episomal supertransfection system. By screening libraries prepared from undifferentiated ES cells it is possible to isolate cDNAs whose products mediate self-renewal. In this case direct selection can be applied for colony formation in the absence of LIF. For cDNAs whose products direct differentiation, however, it may be necessary either to screen pools through several rounds or to incorporate an inducible promoter into the episome.

Recently, several improved protocols for in vitro differentiation of ES cells have been reported, which promote efficient generation of, for example, haematopoietic cells (33), neurons (34) or cardiomyocytes (35). The episomal expression strategy of the invention can be applied for gain-of-function assays and screens during these differentiation programmes. It can also be used for loss-of-function analyses via overexpression of anti-sense RNA or dominant-negative mutants. Combination of these differentiation systems with the episomal expression system provides powerful tools for analysing cell determination and differentiation events.

The invention further provides, in an additional aspect an improvement on the above expression system.

A method of the additional aspect, for expressing a product DNA in a cell, comprises:
(a) (i) transfecting a population of cells with a first vector that expresses a replication factor; or
   (ii) otherwise obtaining a population of cells that express or will express the replication factor;
(b) transfecting cells in the population of cells with a second vector, wherein
   (i) the second vector contains a first DNA in operative combination with a promoter for expression of the first DNA; and
   (ii) extrachromosomal replication of the second vector is dependant upon presence within the cell of the replication factor;
(c) isolating cells that express the first DNA to form a first isolated population of cells;
(d) culturing the first isolated population of cells;
(e) isolating, from the culture of (d), cells that do not express the first DNA to form a second isolated population of cells; and
(f) expressing the product DNA in a cell of the second isolated population of cells.

This aspect also provides a method of expressing a product DNA in a cell, comprising:
(a) (i) transfecting a cell with a first vector that expresses a replication factor; or
   (ii) otherwise obtaining a cell that expresses or will express the replication factor;
(b) transfecting the cell with a second vector, wherein
   (i) the second vector contains a first DNA in operative combination with a promoter for expression of the first DNA; and
   (ii) extrachromosomal replication of the second vector is dependant upon presence within the cell of the replication factor;
(c) obtaining cells from the transfected cell of (b) and isolating a cell that expresses the first DNA to form a first isolated cell;
(d) culturing the first isolated cell to form a culture comprising a plurality of cells;
(e) isolating, from the culture of (d), a cell that does not express the first DNA to form a second isolated cell; and
(f) expressing the product DNA in a cell of the second isolated population of cells.

In this additional aspect, there is the advantage that a cell or population of cells can be obtained which will support transfection by a second vector and its subsequent episomal maintenance at a higher efficiency than before.

The product DNA can be expressed by the step of transfecting a cell of the second isolated population of cells with a third vector containing the product DNA operatively linked to a promoter for expression of the product DNA, extrachromosomal replication of the third vector being dependent upon presence of the replication factor. Thus, having obtained a population that is more susceptible to supertransfection this population can be used for expression of DNA in cells by supertransfection with an appropriate vector, say as part of a functional DNA screen.

The invention also provides a method of selecting a cell or a cell population, comprising:
(a) (i) transfecting a population of cells with a first vector that expresses a replication factor; or
   (ii) otherwise obtaining a population of cells that express or will express the replication factor;
(b) transfecting cells in the population of cells with a second vector, wherein
   (i) the second vector contains a first DNA in operative combination with a promoter for expression of the first DNA; and
   (ii) extrachromosomal replication of the second vector is dependent upon presence within the cell of the replication factor;
(c) isolating a cell or cells that express the first DNA to form a first isolated cell or first isolated population of cells;
(d) culturing the first isolated cell or first isolated population of cells;
(e) subsequently isolating, from the culture of (d), a cell or cells that do not express the first DNA to form a second isolated cell or second isolated population of cells; and
(f) selecting the cell or cell population from the second isolated cell or second isolated population of cells.

Step (d) preferably comprises culturing until the second vector is no longer episomally maintained in cells of the first isolated population—hence removing expression of marker which is episomal. This can be done with a first DNA which encodes a selectable marker and by, in step (d), culturing in the absence of selection for the selectable marker until the second vector is no longer episomally maintained in cells of the first isolated population, preferably culturing until the second vector is no longer episomally maintained in substantially all of the cells. The culture time varies, though culture of cells for 2 or more days, or 4 days or more may be sufficient in many instances. After this period of culture without selection it is found that cells lose the episome, though there is contaminating expression from DNA which has integrated. The method enables removal of this contamination as it can be identified and a further selection or separation carried out, and successive rounds of the method carried out by the inventors has produced a population which can be transfected with an efficiency of 2%, others give 3% and high efficiency.

Step (c) hence preferably comprises selecting for cells that express the first DNA. Generally, the first DNA codes for a marker, especially a selectable marker. One suitable selectable marker protects the cells from the effect of a cytotoxic agent added to cell culture medium; step (c) then comprises isolating cells that express the marker by adding the cytotoxic agent to culture medium and retaining cells that survive. If the selectable marker is drug resistance, say resistance to puromycin then culture in the presence of puromycin enables selection.

Another useful selectable marker is a cell surface antigen; cells can be separated by e.g. FACS into those that express the antigen and those which do not, e.g using an antibody that binds to the cell surface antigen. This marker has the advantage that cells not expressing the marker can be separated without killing them.

In a preferred embodiment of the invention described in an example below the second vector contains the first DNA that encodes a first marker and also contains a second DNA that encodes a second marker. In step (c) there is selection for cells that express the first DNA and in step (e) selection for cells that don't express the first DNA by selecting for cells that don't express the second DNA.

Another advantage of the invention is that it enables generation of a clone of cells, especially of pluripotent cells such as ES cells, which can be transfected with the second vector at high efficiency. This can be achieved by isolating cells that don't express the first DNA by picking an individual cell from the culture of (d); growing the cell into a clonal population; dividing the clonal population into first and second sub-populations: transfecting the first sub-population with the second vector but not transfecting the second sub-population with the second vector; and isolating the cells provided that cells of the first sub-population express the marker and that no cells of the second sub-population express the marker, or by isolating cells that don't express the first DNA by picking an individual cell from the culture of (d); growing the cell into a clonal population; dividing the clonal population into first and second sub-populations; transfecting the first sub-population with the second vector wherein the first DNA encodes resistance to a cytotoxic agent but not transfecting the second sub-population with the second vector; and isolating the cells provided that cells of the first sub-population are resistant to the cytotoxic agent and that no cells of the second sub-population are resistant to the cytotoxic agent.

In this way a clone is retained which has been transfected and then lost the episome but which has not kept the DNA as integrated DNA. Again, a clone is obtained that is susceptible of transfection at a higher efficiency and this has been found to increase over successive rounds of selection.

Still further the invention provides a method of obtaining a cell, comprising:—

(1) obtaining a cell which expresses a replication factor;

(2)transfecting the cell with a vector which requires the presence of the replication factor to be maintained episomally within the cell;

(3) expanding the cell into a plurality of cells; and

(4) selecting for a cell in the plurality of cells (i) which maintains the vector episomally, and (ii) in which the vector has not integrated into chromosomal DNA of the cell.

Yet further the invention provides a method of obtaining a cell, comprising:—

(1) obtaining a population of cells which express a replication factor;

(2) transfecting cells of the population with a vector which requires the presence of the replication factor to be maintained episomally within the cells;

(3) culturing the cells to obtain a plurality of cells; and

(4) selecting for a cell in the plurality of cells (i) which maintains the vector episomally, and (ii) in which the vector has not integrated into chromosomal DNA of the cell.

The obtaining of (1) can be carried out by transfecting the cell with a first vector that expresses a replication factor, and the culturing of (3) can comprise maintaining the cells in culture for at least 2 generations.

In a particular method of the invention, the transfecting of (2) comprises transfecting the cells with a second vector that contains a first DNA encoding a marker and the selecting of (4) comprises isolating cells that express the first DNA by isolating cells which have made the marker, culturing those isolated cells until the cells no longer maintain the vector episomally and then selecting for cells which do not express the first DNA. In examples, a period of 4 days has been sufficient for substantially all cells to lose the second vector as an episome.

Alternatively, the selecting of (4) comprises isolating cells in which the vector is not integrated and then isolating cells which maintain the vector episomally. It is also optional to select the cells according to step (4) and then repeat steps (2) to (4) with that selected cell as the cell which is transfected in step (2).

In using the invention, cells are identified those that are susceptible of being supertransfected but in which the vector DNA does not integrate. When the process is repeated several times, at each step cells which allow the DNA to integrate are eliminated and there is an increase in the proportion of cells in which supertransfection can successfully be carried out.

Transfecting means carrying out a process designed to transfect a cell e.g. with a vector so that the vector is taken up and expressed. Successful transfection is usually reported by DNA in the vector which codes for a marker, e.g. green fluorescent protein or drug resistance. Typically transfection is carried out on a plurality of cells in culture and not all cells take up and express the nucleic acid, usually DNA, of the vector. In prior art transfections of the type described herein the rate of take-up is typically about 1% in cells that are identified as competent to take-up the DNA and maintain it episomally. For green fluorescent protein, successful take-up and expression of DNA encoding this marker is observed by fluorescence of the cell. For drug resistance, successful take-up and expression is demonstrated by a cell which is resistant to the drug in question e.g. when the drug is added to medium in which the cell is cultured, cells not expressing the drug resistance die.

A vector of the additional aspect comprises (a) an origin of replication, (b) DNA encoding a first selectable marker operatively linked to a promoter and (c) DNA encoding a second selectable marker operatively linked to a promoter, wherein the first selectable marker is resistance to a cytotoxic agent and the second selectable marker is a cell surface antigen and wherein the vector does not include DNA encoding a replication factor that binds to the origin of replication.

The first selectable marker can be resistance to a drug such as puromycin, G418, hygromycin or another cytotoxic and/or antibiotic agent that kills mammallian ES cells. Culture in the presence of the drug enables section for cells expressing the marker. The second selectable marker can be a fluorescent protein, such as gfp, or a cell surface antigen to which an antibody binds so that cells (i) expressing and (ii) not expressing the marker can be separated.

The invention further provides a pluripotent cell, especially an ES cell, expressing a replication factor and capable of being transfected with a vector, wherein replication of the vector is dependent upon presence within the cell of the replication factor, at an efficiency of 2% or higher, preferably 2.5% or higher, more preferably 3% or higher. The invention still further provides cell lines, obtained according to the methods of the additional aspects of the invention.

Particular cells are mouse and human pluripotent cells, preferably ES cells, obtainable by carrying out a method of the additional aspect of the invention. Preferred cells can be transfected by a vector of the additional aspect at an efficiency of at least 2%, more preferably at least 2.5% even more preferably at least 3%. In use of this technology, once a cell or population cells is established that can be transfected at this high efficiency it is used for functional gene screening, e.g. it is transfected with a vector whose replication and episomal maintenance is dependent upon existing production of a replication factor in the cell and which encodes a polypeptide (or other DNA product) to be screened.

### BRIEF DESCRIPTION OF THE DRAWINGS

The invention is now described with reference to the accompanying drawings in which:

FIG. 1 shows the structure of the episomal expression vector pHPCAG;

FIG. 2 shows supertransfection efficiency of pHPCAG in MG1.19 ES cells;

FIG. 3 shows DNA hybridisation analysis of Hirt supernatants from supertransfectants;

FIG. 4 shows the effect of vector size on supertransfection efficiency;

FIG. 5 shows expression of β-galactosidase in MG1.19 transfectants;

FIG. 6 shows the restriction pattern of plasmid DNAs recovered from pHPCAG-lacZ supertransfectant clone;

FIG. 7 shows induction of differentiation by expression of STAT3F in MG 1.19 ES cells;

FIG. 8 shows co-supertransfection of STAT3F with wild type STAT expression vectors; and

FIG. 9 shows a vector for use in an assay of the invention;

### DETAILED DESCRIPTION OF THE DRAWINGS

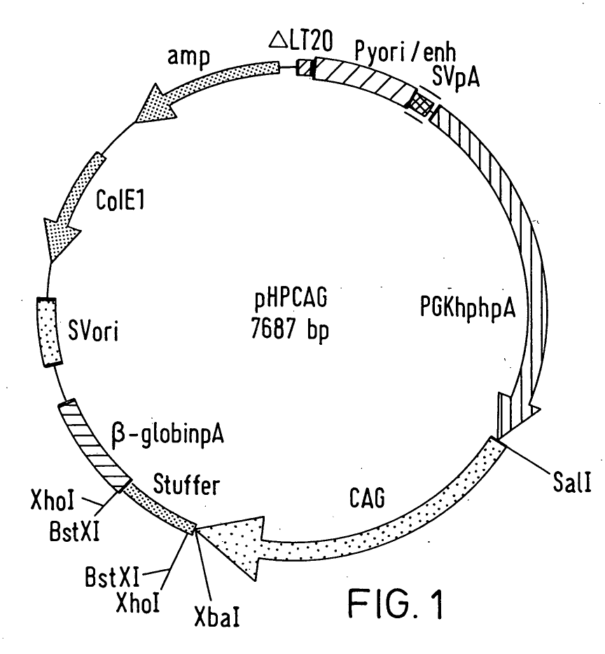

FIG. 1 shows the structure of the episomal expression vector pHPCAG. cDNAs can be introduced between two BstXI sites using BstXI adaptors. Abbreviations: ΔLT20: deleted polyoma large T expression cassette LT20; Pyori/enh: mouse polyoma virus replication origin and mouse polyoma mutant enhancer derived from F101 strain; SVpA: SV40 polyA addition signal; PGKhphpA: hygromycin B phosphotransferase gene expression cassette with mouse phosphoglycerokinase-1 (PGK) promoter and polyA addition signal; CAG: combined CAG expression unit; β-globinpA: rabbit β-globin polyA addition signal; SVori: SV40 replication origin; ColE1ori: ColE1 replication origin; amp: E. coli β-lactamase gene conferring resistance to ampicillin.

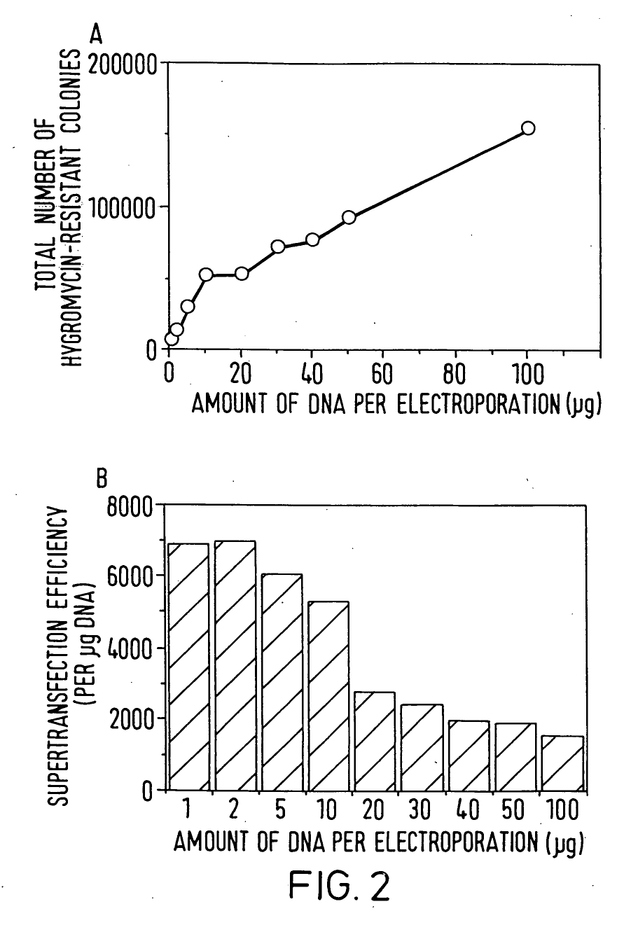

FIG. 2 shows supertransfection efficiency of pHPCAG in MG1.19 ES cells.

(A) shows numbers of transfectant colonies per microgram of pHPCAG DNA. 5×10⁶ MG1.19 ES cells were supertransfected with the indicated amounts of supercoiled pHPCAG followed by selection with hygromycin B for 8 days. The resulting number of drug-resistant colonies were scored and efficiency per μg DNA calculated.

(B) shows total numbers of transfectant colonies plotted against total amount of plasmid DNA.

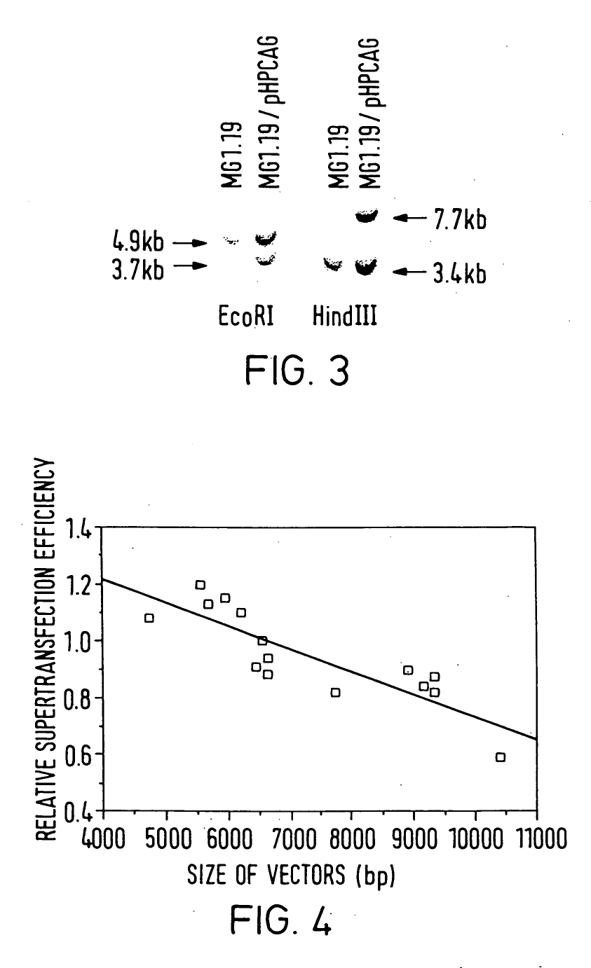

FIG. 3 shows DNA hybridisation analysis of Hirt supernatants from supertransfectants. Hirt supernatants were prepared from 5×10⁶ parental MG1.19 cells and pooled pHPCAG supertransfectants. 1/20 of each sample was digested with either Eco RI or HindIII and analyzed by filter hybridisation using a 344 bp Sca I-SspI fragment from pUC19 which is common to both pMGD20neo and pHPCAG.

FIG. 4 shows the effect of vector size on supertransfection efficiency. 20 μg of each of the supercoiled vectors pLT20ΔNdeIhph (4.7), pLT20ΔBstXIhph (5.5), pLT20ΔAlwNIhph (5.6), pLT20ΔSacIhph (5.9), ptkp (6.2), pSV40e/p (6.4), PGKhphΔLT20 (6.5), pmPGKp (6.6), phBAp (6.6), pHPCAG (7.7), ptkp-lacZ (8.9), pSV40e/p-lacZ (9.1), pmPGKp-lacZ (9.3), phBAp-lacZ (9.3), and pHPCAG-lacZ (10.4) were individually supertransfected into 5×10⁶ MG1.19 ES cells. The resulting numbers of hygromycin B resistant colonies were scored after 8 days. Transfection efficiencies are normalised relative PGKhphΔLT20.

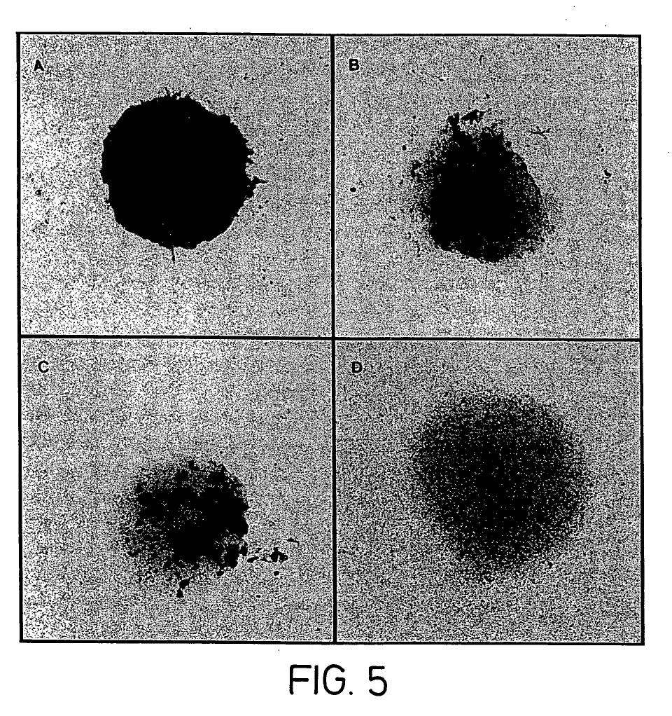

FIG. 5 shows expression of β-galactosidase in MG1.19 transfectants. Primary colonies were stained with Xgal after 8 days of selection.

(A) shows typical homogeneous staining pattern obtained following supertransfection with supercoiled pHPCAG-lacZ.

(B) shows heterogeneous staining pattern obtained in minority of clones following supertransfection with supercoiled pHPCAG-lacZ.

(C) shows heterogeneous staining pattern typically observed following electroporation of linearized pHPCAG-lacZ and stable integration.

(D) shows rare faint staining pattern obtained after supertransfection with supercoiled pHPCAG-lacZ.

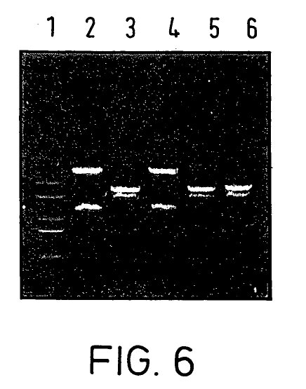

FIG. 6 shows the restriction pattern of plasmid DNAs recovered from pHPCAG-lacZ supertransfectant clone.

A supertransfectant MG1.19 clone carrying pHPCAG-lacZ was cultured for 60 days in the presence of hygromycin B. Hirt DNA was then prepared and electrotransformed into E. coli DH10B cells. Plasmid DNAs were recovered from transformants, digested with EcoRI, resolved by electrophoresis on 1.0% agarose gel and visualised by ethidium bromide staining. Expected fragment sizes: pMGD20neo, 4852 bp and 2884 bp; pHHPCAG-lacZ, 3697 bp, 2810 bp, 783 bp and 397 bp. Lane 1: size marker (1 kb ladder:BRL); lane 2: control pMGD20; lane 3: control pHPCAG-lacZ; lane 4: recovered pMGD20; lane 5,6: recovered pHPCAG-lacZ.

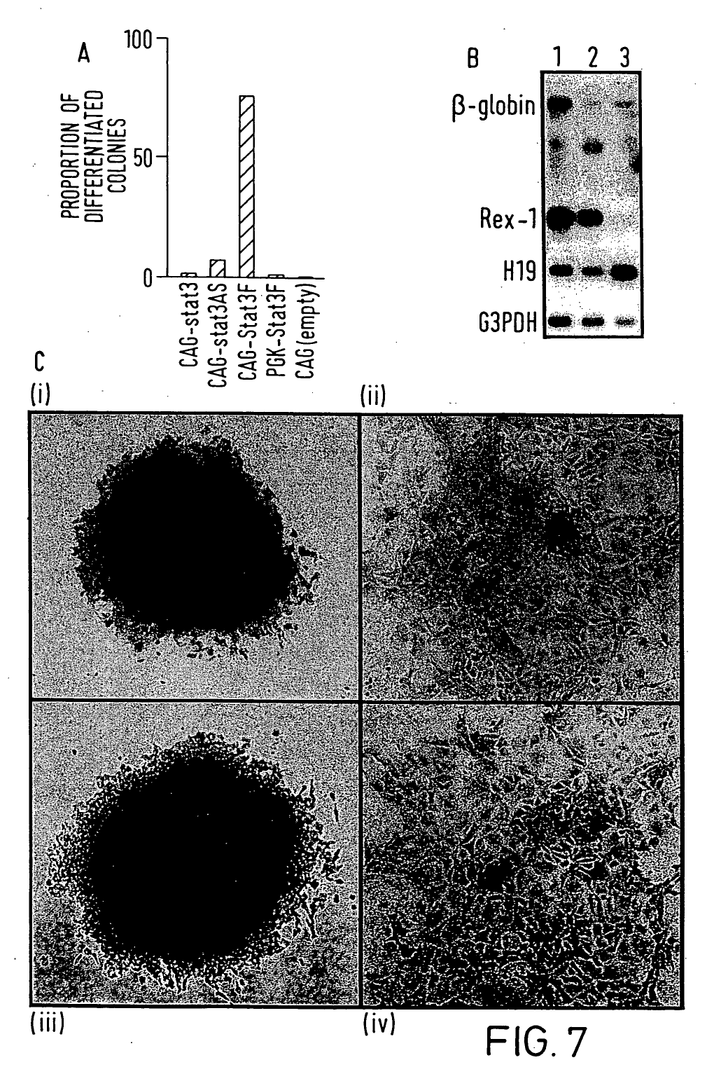

FIG. 7 shows induction of differentiation by expression of STAT3F in MG 1.19 ES cells.

(A) shows proportion of differentiated colonies in LIF-supplemented medium resulting from supertransfection of STAT3, antisense STAT3 and STAT3F expression vectors. Colonies were fixed and stained with Leishman's reagent after 8 days selection and numbers of stem cell colonies and differentiated colonies scored.

(B) shows marker gene expression in STAT3F supertransfectants: Expression of marker genes in pools of MG1.19 cells supertransfected with STAT3 (lane 1), STAT3 antisense (lane 2) and STAT3F (lane 3) expression vectors. Total RNA was prepared after 8 days of selection in LIF-supplemented medium and 5 μg aliquots analyzed by filter hybridisation with β-globin, Rex-1, H19 and G3PDH probes. The β-globin probe detects all transgene mRNA species generated from pHPCAG, including an alternatively spliced product from the antisense construct.

(C) shows photomicrographs of representative colonies 8 days after supertransfection with (i) STAT3, (ii) STAT3F, and (iii) empty expression vectors and selection in the presence of LIF, or, (iv) induction of differentiation by culture in the absence of LIF for 8 days.

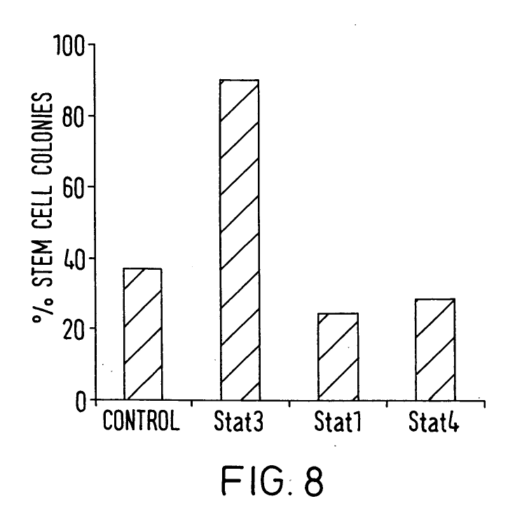

FIG. 8 shows co-supertransfection of STAT3F with wild type STAT expression vectors. Proportions of undifferentiated stem cell colonies generated after co-supertransfection of MG1.19 ES cells with 10 μg pBPCAGGS-STAT3F plus 10 μg pHPCAG vector containing stuffer (control), STAT3, STAT1 or STAT4 inserts. After 8 days selection with 80 μg/ml of hygromycin B plus 20 μg/ml of blasticidin S, colonies were fixed and stained with Leishman's reagent.

The invention is also illustrated in the accompanying sequence listing in which:—

SEQ ID No.s 1 and 2 show a FLAG linker sequence;

SEQ ID No.s 3 and 4 show a [gly₄ser]₂ linker sequence;

SEQ ID No.5 shows DNA encoding a truncated IL6R; and

SEQ ID No.6 shows DNA encoding a modified IL6R.

### EXAMPLES

### Example 1

Materials and Methods

Vector Constructions.

Standard recombinant DNA methods were used to construct all plasmids (10) Plasmid pHPCAG (FIG. 1) was constructed from pMGD20neo (8). The PGKneopolyA sequence was replaced by a hygromycin resistance marker, PGKhphpA, and large T sequences were deleted (see Results). A SalI-ScaI fragment containing the CAG expression unit, a BstXI stuffer sequence, the polyA addition signal derived from the rabbit β-globin gene and an SV40 replication origin (11) was inserted. Coding sequences for β-galactosidase, LIF or interleukin-2 were introduced between the BstXI sites.

For construction of episomal expression vectors with alternative promoters, the SalI-XbaI fragment containing the CAG expression unit in pHPCAG-lacZ was replaced with the 344 bp SV40 enhancer/promoter (SV40e/p), the 466 bp human β-actin promoter (hBA), the 502 bp mouse phosphoglycerate kinase promoter (mPGK) and the 90 bp HSV-tk minimal promoter (tk), resulting in pHPSV40e/p-lacZ, pHPhBA-lacZ, pHPmPGK-lacZ and pHPtk-lacZ, respectively.

Episomal vectors with alternative selection markers were constructed by replacing the PGKhphpA cassette in pHPCAG with the SVbsrpA cassette carrying the E. coli blasticidin S deaminase (bsr) gene derived from pSV2bsr (Waken Seiyaku) or the hCMVzeopA cassette carrying the Streptoalloteichus bleomycin resistant gene (Sh ble) derived from pZeoSV (Invitrogen) to generate pBPCAGGS and pZPCAGGS, respectively.

Cell Culture and Transfection.

MG1.19 ES cells are derivatives of the CCE line which stably maintain around 20 episomal copies of pMGDneo (8). They were maintained on gelatin-coated plates in Glasgow modified Eagle's medium (GMEM, Gibco-BRL) supplemented with 10% fetal calf serum, 0.1 mM β-mercaptoethanol, non-essential amino acids, 200 μg/ml G418, and 100 U/ml LIF produced in COS-7 cells (11,12). For supertransfection, routinely, 5×10⁶ MG1.19 cells were suspended in 800 μl of PBS, incubated with 20 μg of supercoiled vector DNA for 10 min on ice, and electroporated at 200 V/960 μF using a Bio-Rad gene pulser. Cells were transferred into gelatinized plates and allowed to recover overnight before addition of appropriate selection agent. Histochemical staining for β-galactosidase was carried out with 5-bromo-4-chloro-3-indolyl β-D-galactopyranoside (X-gal) (13), and β-galactosidase activity was measured by incubation of cell extracts with o-nitrophenyl-β-D-galactopyranoside (ONPG). Differentiation was induced in monolayer culture as described (12).

Analysis of Episomal Vectors in the Supertransfectants.

Hirt supernatants were prepared as described (14). For amplification of recovered episomal vectors, electrocompetent E. coli DH10B cells were transformed by electroporation at 2500 V/25 μF/200½.

Results

Construction of an Episomal Expression Vector.

Polyoma-based plasmids have recently been reported to be competent for episomal propagation in ES cells (8). The plasmid pMGD20neo contains a modified large T expression unit called LT20, the viral origin of replication (On), and the PGKneopA cassette as a selectable marker. This plasmid can be maintained as an extrachromosomal element in wild-type ES cells. It can be modified to include a cDNA expression unit (9). However, the low frequency of conventional stable transfection of ES cells (approximately 1×10⁻⁵) remains a limiting feature. Furthermore, episomal propagation only occurs in 10-15% of primary transfectants (8,9).

A second plasmid has been described which can be maintained as an episome only in ES cells which independently express the large T protein (8). This plasmid, PGKhphΔLT20, contains LT20 with a large deletion in its coding sequence, Ori, and PGKhphpA as a selectable marker. When introduced into a cell line such as MG1.19, in which episomal maintenance of pMGDneo has already been established, the yield of hygromycin B resistant stable transfectants is extremely high. This phenomenon of supertransfection is presumed to arise from the pre-existence of large T protein in the recipient cells.

Size of Vector

PGKhphΔLT20 retains part of the large T coding sequence. We made a series of deletions in the ΔLT20 sequence to minimize the vector size and thereby increase the capacity for inserts and reduce potential bias in the construction and screening of cDNA libraries. The supertransfection efficiency of four derivative plasmids was then compared in MG1.19 cells. All showed comparable supertransfection efficiency to PGKhphΔLT20 (data not shown). The smallest, pLT20ANdeIhph, has a deletion of 2953 bp, yielding an episomal vector backbone of only 4.7 kb.

Expression Unit

Into this minimal episomal vector we introduced a cDNA expression unit. Transcriptional initiation signals are supplied by the CAG cassette (11), which comprises the human cytomegalovirus immediate early enhancer, a 1 kb fragment of the chicken β-actin gene (promoter, non-coding first exon and first intron), and a splice acceptor derived from the rabbit β-globin gene. This combination has been shown to direct strong expression of cDNAs in undifferentiated stem cells. The resulting expression vector, pHPCAG (FIG. 1), contains the CAG sequences followed by the BstXI stuffer sequence derived from pCDM8 as a cDNA cloning site, and a polyA addition signal derived from the rabbit β-globin gene. In addition the plasmid contains the PGKhphpA (15) cassette for hygromycin selection of ES cell transfectants, the polyoma Ori with pyF101-derived mutant enhancer element (16) for stable episomal replication in cells expressing polyoma large T protein, and the β-lactamase (amp) gene and prokaryotic replication origin for amplification in E. coli. The SV40 Ori is also present to allow for transient episomal replication in mammalian host cells expressing SV40 largeT, such as COS cells (17).

Characterization of Supertransfection.

The parameters of supertransfection with pHPCAG and derivatives were investigated. First, 5×10⁶ MG1.19 cells were electroporated with various amount of supercoiled pHPCAG, selected in medium containing 80 μg/ml of hygromycin B for 8 days, and the number of stem cell colonies scored after Leishman's staining (12). Although the highest efficiency per pg DNA was observed with minimum amounts (1-2 μg) of vector DNA (FIG. 2B), the total yield of hygromycin B resistant colonies increased with increasing amount of plasmid (FIG. 2A). Saturation was not reached over the range of plasmid concentrations tested. With 100 μg plasmid DNA, 150,000 hygromycin B-resistant colonies were obtained, representing 3% of total treated cells. Disablement for episomal replication by linearisation of pHPCAG prior to electroporation reduced this transfection efficiency to less than 0.01%.

Next, increasing numbers of MG 1.19 cells were subjected to electroporation with 100 μg of pHPCAG DNA. Comparable stable transfection efficiencies in the range 3-6% were obtained with up to 2.5×10⁷ cells.

The copy number of pHPCAG in the supertransfectants was analyzed by preparation of Hirt supernatants followed by filter hybridisation. This analysis revealed that supertransfected cells carried approximately 20 copies each of pMGDneo and pHPCAG (FIG. 3).

These data demonstrate that the efficiency of supertransfection with pHPCAG is extremely high. However, episomal vectors can be limited in their capacity for inserts because increased size may cause inefficient replication or instability. To investigate this issue in the ES cell system, episomal vectors of different size were supertransfected into MG 1.19 cells. The numbers of supertransfectant colonies were scored and plotted against vector size (FIG. 4). These data indicate that there is a progressive reduction in transfection efficiency with increasing plasmid size. In particular, the largest plasmid tested, a derivative of pHPCAG with a 3 kb lacZ insert (total size 10.4 kb) showed a 50% reduction in colony number. However, that this may not be due entirely to the size of the plesmid because the very high levels of β-galactosidase expression may exert some toxic effects (see below).

lacZ Expression in Supertransfectants.

To evaluate the level and pattern of expression of transgenes from pHPCAG, the E. coli β-galactosidase (lacZ) gene was introduced into this vector. The resulting vector, pHPCAG-lacZ, was introduced into MG1.19 cells and supertransfectants isolated by selection with 80 μg/ml of hygromycin B for 8 days. The number of colonies isolated was 50% of the number obtained in a parallel supertransfection with pHPCAG (see above). The colonies were smaller and many of the cells showed an abnormal spindle-shaped morphology. These effects were not observed with several other inserts in pHPCAG and are suggestive of a toxic effect of the high level lacZ expression. The primary supertransfectants were stained with X-gal and the staining pattern examined under phase-contrast microscopy. Staining was detectable after 5 minutes incubation and was intense by 1 hour. This level of β-galactosidase activity is significantly higher than we have observed from a variety of integrated expression constructs.

Approximately 80% of supertransfectant colonies showed ubiquitous expression (>90% cell positive) as shown in FIG. 5-A (i). Of the remainder, 15% showed heterogeneous expression (FIG. 5-A (ii)), and 5% showed little or no staining (FIG. 5-A (iv)). The latter two classes are likely to arise as a result of vector integration which occurs in up to 20% of supertransfectants (8). In transfectants derived by electroporation of linearized pHPCAG-lacZ into MG1.19 cells (which results in vector integration in the majority of clones), only 15% of colonies showed homogeneous staining whereas 70% of colonies stained heterogeneously (FIG. 5-A (iii)), and 15% showed no expression.

Analysis of expanded clones from each class of transfectant established that this difference in expression characteristics was stable. Twelve of 13 expanded supertransfectants expressed lacZ homogeneously. In contrast, only 4 out of 24 clones derived using linearized vector showed homogeneous expression. This is consistent with our previous observations on integrated expression constructs in ES cells. In fact the CAG unit gives a significantly higher frequency of colonies which show stable ubiquitous expression than other promoters we have examined.

The difference in staining pattern between episomally maintained and integrated vectors indicates that the former escape modifying influences arising from integration and reliably give full activity of the expression unit.

Comparison of Expression with Various Promoters on Episomal Vector.

An ability reliably to generate predetermined levels of expression would be a important attribute for a transgene expression system. The previous observations suggested that episomal vectors offered potential to achieve unmodified expression. Various promoters with different strengths in undifferentiated stem cells were therefore introduced into the episomal vector by replacing the CAG expression unit of pHPCAG-lacZ. Expression of the lacZ reporter was then assayed in both transient and stable supertransfectants (Table 1). The relative ratio of β-galactosidase activity obtained from the SV40 enhancer/promoter complex, the human β-actin promoter, the mouse PGK-1 promoter and the HSV-tk minimal promoter in transient transfectant was retained in stable supertransfectants. The CAG expression unit showed strongest activity in the tested constructs in both transient and stable transfectants. In this case, however, the relative ratio in transient transfectants, 19 times higher than SV40, was significantly reduced in stable transfectants. This may arise from an elimination of strong expressants due to a toxic effect of high lacZ expression (see above). A reduced number of supertransfectants and smaller size of colonies was observed only with the CAG vector.

Stability of Supertransfected Episomal Expression Vector During Long-Term Culture and Differentiation of Host Cells.

A critical limitation of previously described episomal vectors is their instability during long-term culture. Many episomal vectors undergo integration into the host genome after long-term culture, resulting in a reduction in expression and inability to recover transgenes by preparing Hirt supernatants. To test the stability of the supertransfection system, four pHPCAG-lacZ supertransfectant clones were cultured for 60 days (approximately 90 generations) under continuous selection with 80 μg/ml of hygromycin B. Three of the four clones maintained relatively constant levels of β-galactosidase activity determined by ONPG assay and uniform expression as revealed by Xgal staining. The fourth clone showed unstable and variegated expression, as commonly observed on vector integration. Hirt supernatants were prepared from one of the stably expressing clones at the end of the 60 day culture period. Filter hybridization analysis of the Hirt DNA indicated that the ES cells carried approximately 20 copies of pMGD20 and 5 copies of pHPCAG-lacZ per cell (data not shown). The lower copy number of pHPCAG-lacZ may be due to its larger size and/or the toxic effect of strong lacZ expression. The Hirt DNA was transformed into E. colior further analysis. Of the bacterial transformants, 20% carried pHPCAG-lacZ and the remainder carried pMGDneo20, in good agreement with the hybridization data. Restriction mapping showed no evidence of rearrangement in either plasmid (FIG. 6).

In the experiment above, cells were maintained under selection with hygromycin B. In the absence of selection pressure, supertransfectant clones lost expression of β-galactosidase over several passages in culture. This might indicate an intrinsic instability of supertransfected episomal vectors. However, it could also reflect a selective disadvantage for ES cells which express high levels of β-galactosidase. It is noteworthy in this regard that the primary episome, pMGD20neo, is stable in the absence of selection (8).

Stability of expression from pHPCAG-lacZ during the in vitro differentiation of ES cells was also analyzed. Differentiation was induced in three ways: withdrawal of LIF; exposure to retinoic acid; and treatment with 3-methoxybenzamide (18). After 6 days the differentiated progeny stained ubiquitously in all three cases (data not shown).

These data indicate that supertransfected episomal vectors can be maintained in an extrachromosomal state and direct strong expression of transgenes during long-term self-renewal and differentiation in vitro.

Production and Secretion of the Cytokine LIF from an Episomal ES Cell Expression Vector.

The pHPCAG-lacZ plasmid can efficiently direct strong and homogeneous expression of the cytoplasmic lacZ reporter gene. We next investigated expression of a secreted molecule, the cytokine LIF. LIF is an essential supplement to ES cell culture medium because it inhibits differentiation of the stem cells (19,20). Expression of LIF can readily be assayed by formation of stem cell colonies in media lacking the cytokine.

Episomal vectors for expression of another cytokine, interleukin-2 (which has no effect on ES cell phenotype), and for LIF were electroporated in parallel into MG1.19 cells. The cells were seeded at low density (1.5×10⁴ and 5×10³ cells per 90 mm plate) to avoid the rescue effect which arises from the production of LIF by differentiated ES cell progeny (21), and cultured with 80 μg/ml of hygromycin B for 8 days. pHPCAG-il2 generated large numbers of stem cell colonies in medium supplemented with LIF, but none in the absence of LIF. pHPCAG-lif in contrast produced comparable numbers of healthy stem cell colonies in both the presence and absence of exogenous LIF (Table 2). These colonies could be expanded and propagated without LIF-supplementation of the medium. These data confirm previous observations that increased autocrine expression of LIF renders ES cells factor-independent (22) and establish that secreted proteins are produced efficiently and stably by this episomal expression system.

Co-Supertransfection of Episomal Vectors.

Introduction of two or more different transgenes into cells is often required for analysis of protein interactions and/or co-operative function. The poor efficiency of homogeneous expression in conventional transfectants is a major obstacle for such investigations in ES cells. To test the possibility that the episomal approach could be applied to co-express multiple cDNAs, we constructed episomal expression vectors with different selection markers. Co-supertransfection of episomal vectors was then assessed.

The basic episomal expression vector pHPCAG carries the hygromycin phosphotransferase gene driven by mouse PGK-1 promoter (PGKhphpA). We prepared episomal vectors which carry the zeocin-resistance gene driven by the human cytomegalovirus immediate-early promoter (pZPCAG), or the blasticidin S-resistance gene driven by the SV40 enhancer/promoter (pBPCAG) by substitution of the PGKhphpA cassette in pHPCAG. These vectors were supertransfected into MG1.19 cells followed by 8 days selection with the appropriate antibiotic. Comparison of the numbers of resulting drug-resistant colonies (Table 3) revealed that these selection systems are slightly less efficient than hygromycin B selection but nonetheless enable large numbers of supertransfectants to be isolated.

ES cells harbouring two different episomal vectors can be isolated by repeated supertransfection. Supertransfectants carrying pHPCAG can be transfected again with pBPCAG or pZPCAG, with comparable efficiency to the original supertransfection into MG1.19 ES cells (data not shown). This should allow establishment of efficient screens for assaying functional interactions between gene products.

The effects of co-electoporation of supertransfection vectors were also investigated. pHPCAG (10 μg) and pBPCAG (10 μg) were co-electroporated into 5×10⁶ MG1.19 cells. Cells were selected in hygromycin B or blasticidin S only, or both, for 8 days and the number of drug-resistant colonies scored in each case. The numbers of hygromycin or blasticidin S single-resistant colonies were 39,000 and 13,000, respectively, while the number of double-resistant colonies was 1,200. Thus the apparent efficiency of incorporation of both plasmids was less than 10%. Similar results were obtained on co-supertransfection of pHPCAG and pZPCAG (not shown).

These data suggest that the majority of supertransfectants incorporate only one plasmid under these electroporation conditions. This is significant for application of the episomal system to functional cDNA library screening.

### Example 2

The effects of overexpression of a large number of transgenes in ES cells were investigated by construction of vectors based on pHPCAG and including a DNA insert coding for the transgene being investigated. 5×10⁶ ES MG1.19 cells were supertransfected with 20 μg of expression vectors and selected with 80 μg/ml of hygromycin B for 8 days. The numbers of drug-resistant colonies were counted and normalised relative to numbers obtained with empty vector. The results are shown in Table 4.

### Example 3

Inhibition of STAT3 Activation Blocks Self-Renewal and Promotes Differentiation

To assess directly the requirement for STAT3 activation in ES cell self-renewal, we exploited a dominant interfering mutant form of STAT3, STAT3F. In this mutant (Minami et al., 1996), the tyrosine residue at amino acid position 705 is mutated to phenylalanine. Phosphorylation of Tyr705 is required for dimerization and nuclear translocation. When expressed at high level, STAT3F has been shown to block the activation of endogenous STAT3 in various cell types, possibly by titrating out receptor docking sites (Fukada et al., 1996; Minami et al., 1996; Nakajima et al., 1996; Bonni et al., 1997; Ihara et al., 1997).

Using conventional transfection approaches we were unable to recover ES cell transfectants showing stable high level expression of STAT3F. In parallel experiments, however, transfection of the LIF-independent embryonal carcinoma cell line P19 yielded multiple expressing clones. This suggested that blockade of STAT3 activation in ES cells specifically resulted in cell death, growth arrest or differentiation. The transfection and expression strategy of the invention was therefore adopted to enable characterisation of the consequences of STAT3F expression.

The STAT3F mutant cDNA was introduced into the supertransfection vector pHPCAG. The wild type STAT3 coding sequence was also introduced, in both sense and antisense orientations. The three constructs were electroporated into MG1.19 cells which harbour a large T expression plasmid and can be supertransfected with constructs containing the polyoma origin (Gassmann et al., 1995). Supertransfectants were isolated by selection in hygromycin B for 8 days in the presence of LIF. Colonies were fixed, stained with Leishman's reagent, counted, and scored for the presence of stem cells and differentiated cells. More than 95% of colonies obtained following supertransfection with control or wild type STAT3 vector were stem cell colonies (FIG. 7A). A modest increase in the proportion of differentiated colonies was obtained with the antisense construct. The STAT3F vector, however, yielded predominantly differentiated colonies. A decrease in total number of colonies was also observed after supertransfection with STAT3F. This may reflect an early onset of differentiation which would produce very small clones that would not be scored. Alternatively, very high levels of STAT3F expression may also be toxic, though this has not been reported in other cell types. Morphologically, the differentiated STAT3F colonies closely resembled the differentiated colonies generated on culture of ES cells in the absence of LIF (FIG. 7C). Various other cDNAs have been expressed in ES cells using this system, with little or no effect on differentiation (data not shown). This suggested that the effect on differentiation was specifically attributable to expression of STAT3F.

The differentiation induced by expression of STAT3F was examined further by expression analysis of the marker genes rex1 and H19. Rex-1 mRNA, which is specifically expressed in undifferentiated stem cells, was down regulated in STAT3F supertransfectants. In contrast, H19 RNA which is found at low levels in stem cells but is upregulated during differentiation, was increased (FIG. 7B). A similar pattern of gene regulation is observed during differentiation of ES cells induced by withdrawal of LIF. These data confirm that the morphological differentiation triggered by STAT3F is accompanied by reprogramming of gene expression.

STAT3F was also expressed from the mouse phosphoglycerate kinase (pgk-1) promoter in the episomal vector pHPPGK. This vector gives at least 10-fold lower expression than pHPCAG (data not shown). In this case, there was no significant effect on either colony number or differentiation status of MG 1.19 supertransfectants. A critical level of expression of the dominant interfering mutant therefore appears necessary to block self-renewal.

Effect of STAT3F on Self-Renewal is Suppressed by Co-Expression of STAT3

To test whether the induction of differentiation by expression of STAT3F was due to an inhibition of endogenous STAT3 activity, we attempted to rescue the stem cell phenotype by co-expression of wild type STAT3 and also of STAT1 and STAT4. A STAT3F expression vector carrying a blasticidin resistance marker was co-supertransfected into MG1.19 cells with episomal constructs for expression of wild type STATs and hygromycin resistance. Co-supertransfectants were isolated in medium containing both 20 μg/ml of blasticidin S and 80 μg/ml of hygromycin B. The numbers of stem cell and differentiated colonies were scored after 8 days. As shown in FIG. 8, only co-expression of wild type STAT3 restored self-renewal in the presence of STAT3F. Transfection with STAT1 or STAT4 constructs alone had no effect on self-renewal in the absence of STAT3F (not shown) and did . . . not alter differentiation induced by STAT3F. In the case of supertransfection with the CAG promoter STAT1 construct, the total number of colonies (stem+differentiated) recovered was reduced but the relative proportion of stem cell colonies versus differentiated cells was unaltered. This occurred in both the presence and absence of co-expression of STAT3F, and suggests that high level expression of STAT1 may be toxic to ES cells. By using the mouse PGK-1 promoter to drive lower levels of expression comparable numbers of colonies were recovered on transfection with the STAT1 as with the other constructs. In this case, again only the STAT3 construct showed any restoration of stem cell colonies, although to a lower degree than with the high expression CAG vector (not shown). These data indicate that STAT3 has a specific junction in ES cells which cannot be compensated by STAT1 or STAT4.

### Example 4

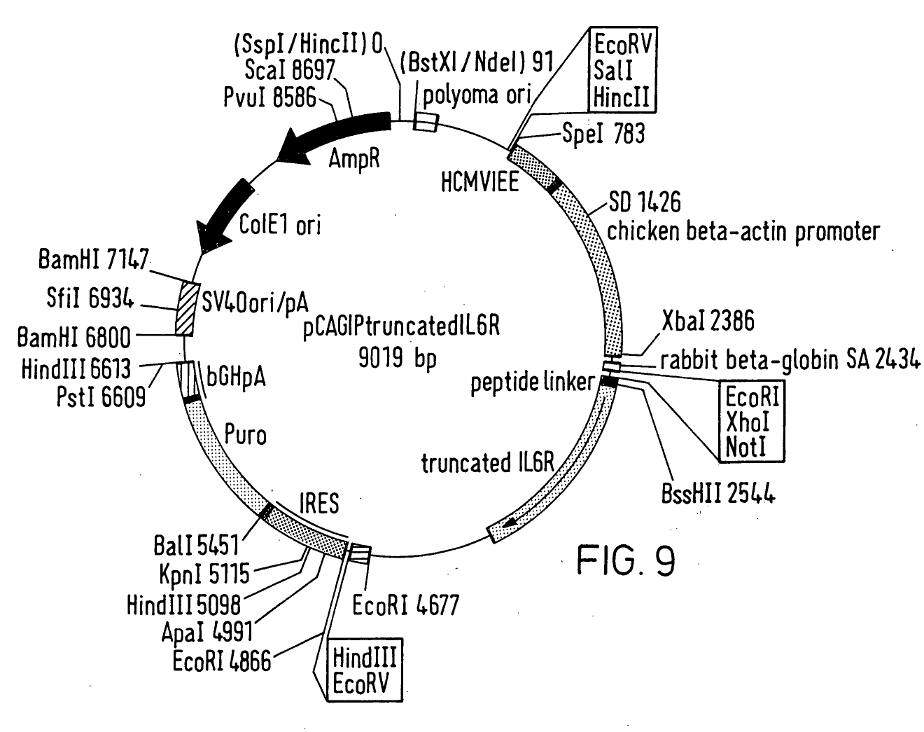

The invention is also used in a strategy for direct selection of genes that code for secreted and cell surface proteins. In one example of this strategy, the basic cloning vector is a truncated form of IL6R that lacks a signal sequence. This vector is described in detail below and shown in FIG. 9. If this truncated IL6R is expressed in ES cells, it is not exported to the cell surface and these cells differentiate when cultured in IL6. However, if the IL6R signal sequence is reconstituted by a signal sequence provided by a cDNA fragments cloned in frame at the 5′ end of the truncated IL6R, the chimaeric receptor is expressed on the surface of ES cells. ES cells containing such chimaeric receptors are thus maintained as undifferentiated colonies when cultured in IL6.

Libraries of short, 5′ cDNA fragments are produced and cloned into a truncated and modified IL6R-based expression vector. ES cells transformed with such libraries express cDNA:IL6R fusion proteins. However, only cDNAs that encode signal sequences confer IL6 responsiveness on ES cells. These cDNAs alone give rise to undifferentiated, proliferating ES cell clones. This strategy therefore provides a direct selection for cDNAs encoding secreted and cell surface proteins.

The chimaeric IL6R is expressed in the episomal expression system described above (or a derivative thereof). This allows drug selection for episomally transformed cells and high level expression of cloned DNA.

To further refine the selection system, ES cells are modified with two targeted mutations:

a) A selectable marker gene, for example the blasticidin resistance gene, is introduced into the OCT-4 locus by standard targeting techniques. Since Oct-4 is expressed in undifferentiated ES cells, the blasticidin resistance gene will be expressed only by undifferentiated colonies. Blasticidin selection therefore is used to decrease background growth by ensuring rapid deletion of differentiating, Oct-4 negative, ES cells.

b) Since ES cells can produce LIF as an autocrine growth factor, ES cells are used in which both copies of the LIFR gene have been disrupted by gene targeting. This eliminates the possibility of LIF-dependent, false positive colonies that might otherwise persist throughout selection in IL6.

Details of Vector Construction:

1). IL6R was cloned into the episomal vector pCAGIP or a derivative (PCAGIPXN, i.e. pCAGIP with a destroyed NotI site). pCAGIP contains an internal ribosome entry site (IRES) and a puromycin resistance gene downstream of its multiple cloning site, resulting in stoichiometric production of cDNA:IL6R fusion proteins in transfected cells under puromycin selection. IL6R in pCAGIP provides a positive control (IL6-responsive functional protein on the cell surface), and the basis of the new vector.

2). To construct the cloning vector, IL6R cDNA was truncated by cleavage with BssHII at nucleotide number 92. This deleted the initiator ATG and sequences encoding the signal sequence.

3). To minimise potential steric interference by cloned proteins with IL6 binding and IL6R function, DNA encoding a synthetic flexible linker peptide was then added to the 5′ end of the truncated IL6R. Two alternative linkers have been used: gly gly gly gly ser gly gly gly gly ser and a linker containing the FLAG epitope, gly ser ASP TYR LYS ASP ASP ASP ASP LYS (FLAG epitope in upper case). The sequence of these linkers is shown in SEQ ID No.s 1 and 2, and 3 and 4. In each case, the linker sequence has been cloned in frame with IL6R and has two unique cloning sites (XhoI and NotI) at its 5′ end, allowing the introduction of cDNA libraries, or specific cloned sequences, in a directional manner. The FLAG epitope is recognised by a commercially available monoclonal antibody (M2; available from IBI/Kodak) regardless of its position within a fusion protein, and will thus allow the expression levels of surface protein to be measured directly by immunocytochemistry.

4). Vectors containing each of these linkers and an upstream signal sequence are tested for relative expression level and IL6R-function, as detailed below.

To test the utility of these vectors for selecting proteins expressed at the cell surface, a number of known signal sequences are cloned into each vector. These are tested for surface expression and IL6R function. Signal sequences include those from rat CD4 (a protein with extracellular Ig domains), mouse sek (a receptor tyrosine kinase, with no extracellular Ig domains) and mouse sonic hedgehog (a secreted factor).

ES cells are transfected with vectors bearing candidate signal sequences by lipofection or electroporation, followed by puromycin selection for transfected cells. After overnight growth in the presence of LIF, to maintain the undifferentiated state and proliferation, transfected cells are split into three groups and treated with either 1) LIF, 2) IL6 or 3) neither growth factor. Only cells bearing IL6R brought to the cell surface by a fused signal peptide will proliferate in the presence of IL6. Positive controls include ES cells transfected with wild-type IL6R grown in the absence of LIF and the presence of IL6. Negative controls include empty vector (i.e truncated IL6R with no 5′ insert) grown in the presence of IL6. To determine whether fusion proteins N-terminal to IL6R block signalling (by steric hindrance), the proportion of such cells that express surface protein but fail to proliferate in response to IL6 is deduced by comparing the number of cells expressing the FLAG epitope with the number that give rise to colonies.

Vectors defined by this assay are then used in cDNA library screens. Preferably, sequences corresponding to 5′ ends of cDNAs are generated from full length cDNA libraries and directionally cloned in the screening vector.

### Example 5

Mouse ES cells were engineered to express polyoma large T antigen (PyLT) by transfection with pMGD20neo. Culture in the presence of G418 selected for cells that had taken up the vector. Subsequently, transfection of the PyLT+ES cells was possible at an efficiency of approximately 1% using a second vector containing the Py ori only if the expression level of PyLT was similarly high to the MG1.19 cell line. However, the efficiency of generation of such supertransfectable lines was low (1/114 clonal lines tested by blind colony picking). In separate experiments, cDNA is cloned directionally in place in the second vector and efficient expression achieved by selecting for puromycin—resistant cells (Puromycin resistance being encoded also in the second vector).

An additional strategy was devised to improve the efficiency of generation of new supertransfectable lines. Cells transfected with the first vector (pMGD 20neo) were transfected with a vector encoding both puromycin resistance and green flourescent protein (gfp) using vector designated pPyCAGegfpIP. Selection in puromycin was carried out for a period of 4 days, yielding a population of transfected, puromycin-resistant cells.

This population was then further cultured in the absence of selection, i.e., in the absence of puromycin, for a period of 7 days. This culture condition and time period was designed so that the cells would lose the episomal second vector.

The population was then sorted by FACS into two populations, those expressing gfp and those not expressing gfp, and the population not expressing gfp was retained. The retained cells were then subjected to a further round of transfection with pPyCAGefgpIP, and this transfection was found to have an efficiency of 3.1%. After a further round of selection in puromycin for 4 days and then culture for 7 days in the absence of puromycin, followed by another FACS sort, a population of cells was obtained which were susceptible to transfection at a still higher efficiency than before (i.e. greater than 3.1%).

In a separate method, after culture in the absence of puromycin for 7 days, 18 colonies were picked and expanded into 18 clonal populations. Each population was then divided and each half sub-population transfected with the same second vector (pPyCAGegfpIP) or mock transfected with no DNA. Transfected cells were then plated out and that clone retained only if cells transfected with the second vector showed puromycin resistance and no mock transfected cells showed puromycin resistance (if puromycin resistance is shown by mock transfected cells this indicates that puromycin resistance arises from integration of DNA).

In this experiment six clones were retained out of a total of 18 picked colonies. These showed a transfection of from 1.5-2.5% with no resistant colonies formed in the mock transfected plates.

In the above description scientific publications are referred to under the following reference numbers:
1. Smith, A. G. (1992) Seminars in Cell Biology, 3, 385-399.
2. Evans, M. J. and Kaufman, M. (1981) Nature, 292, 154-156.
3. Martin, G. R. (1981) Proc. Natl. Acad. Sci. USA, 78, 763.4-7638.
4. Doetschman, T. C., Eistetter, H., Katz, M., Schmidt, W. and Kemler, R. (1985) J. Embryol. Exp. Morphol., 87, 27-45.
5. Weiss, M. J. and Orkin, S. H. (1996) J. Clin. Invest., 97, 591-595.
6. Bradley, A., Evans, M. J., Kaufman, M. H. and Robertson, E. (1984) Nature, 309, 255-256.
7. Beddington, R. S. P. and Robertson, E. J. (1989) Development, 105,733-737.
8. Gassmann, M., Donoho, G., Berg, P. (1995) Proc. Natl. Acad. Sci. USA, 92, 1292-1296.
9. Camenisch, G., Gruber, M., Donoho, G., Van Sloun, P., Wenger, R. and Gassmann, M. (1996) Nucleic Acids Research, 24, 3707-3713.
10. Sambrook, J., Fritsch, E. F. and Maniatis, T. (eds.) (1989) Molecular Cloning: A Laboratory Manual. 2nd ed Ed. 3 vols. Cold Spring Harbor Laboratory Press, Cold Spring Harbor, N.Y.
11. Niwa, H., Yamamura, K.-I., Miyazaki, J.-I. (1991) Gene, 108,193-200.
12. Smith, A. G. (1991) J. Tiss. Cult. Meth., 13, 89-94.
13. Beddington, R. S. P., Morgenstem, J., Land, H. and Hogan, A. (1989) Development, 106, 37-46.
14. Hirt, B. J. (1969) J. Mol. Biol., 26, 141-144.
15. te Riele, H., Maandag, E. R., Clarke, A., Hooper, M. and Berns, A. (1990) Nature, 348, 649-651.
16. Fujimura, F. K., Deininger, P. L., Friedmann, T. and Linney, E. (1981) Cell, 23, 809-814.
17. Tsui, L. C., Breitman, M. L., Siminovitch, L. and et al. (1982) Cell, 309, 499-508.
18. Smith, A. G. and Rathjen, P. D. (1991) Sem. Dev. Biol., 2, 317-327.
19. Smith, A. G., Heath, J. K., Donaldson, D. D., Wong, G. G., Moreau, J., Stahl, M. and Rogers, D. (1988) Nature, 336, 688-690.
20. Williams, R. L., Hilton, D. J., Pease, S., Willson, T. A., Stewart, C. L., Gearing, D. P., Wagner, E. F., Metcalf, D., Nicola, N. A. and Gough, N. M. (1988) Nature, 336, 684-687.
21. Rathjen, P. D., Nichols, J., Toth, S., Edwards, D. R., Heath, J. K. and Smith, A. G. (1990) Genes Dev., 4, 2308-2318.
22. Conquet, F., Peyrieras, N., Tiret, L and Brulet, P. (1992) PNAS, 89, 8195-8199.
23. Skarnes, W. C., Auerbach, B. A. and Joyner, A. L. (1992) Genes Dev., 6, 903-918.
24. Friedrich, G. and Soriano, P. (1991) Genes Dev., 5, 1513-1523.
25. Mountford, P., Smith, A. G. (1995) Trend Genet., 11, 179-184.
26. Askew, G. R., Doetschman, T. and Lingrel, J. B. (1993) MCB, 13, 4115-4124.
27. Tucker, K. L., Beard, C., Dausman, J., Jackson-Grusby, L., Laird, P. W., Lei, H., Li, E., Jaenish, R. (1996) Genes & Dev., 10, 1008-1020.
28. Macleod, D., Chariton, J., Mullins, J., Bird, A. (1994) Genes Dev., 8, 2282-2292.
29. Strathdee, C. A., Gavish, H., Shannon, W. R. and Buchwald, M. (1992) Nature, 356, 763-767.
30. Legerski, R. and Peterson, C. (1992) Nature, 359, 70-73.
31. Hayakawa, H., Koike, G., Sekiguchi, M. (1990) J. Mol. Biol., 213, 739-747.
32. Sasaki, K., Watanabe, E., Kawashima, K., Sekine, S., Dohi, T., Oshima, M., Hanai, N., Nishi, T., Hasegawa, M. (1993) J. Biol. Chem., 268, 22782-22787.
33. Nakano, T., Kodama, H., Honjo, T. (1994) Science, 265, 1098-1101.
34. Bain, G., Kitchens, D., Yao, M., Huettner, J. E., Gottlieb, D. I. (1995) Dev. Biol., 168, 342-357.
35. Rohwedel, J., Maltsev, V., Bober, E., Arnold, H. H., Hescheler, J., Wobus, A. M. (1994) Dev. Biol., 164, 87-101.

We have thus described the development of an optimised transfection and expression system which will enable high throughput functional screening of cDNAs in pluripotential mouse embryonic stem (ES) cells and differentiated derivatives. The strategy is based on extrachromosomal vector replication driven by expression of polyoma large T protein. When a vector containing a polyoma origin of replication is introduced into an ES cell line that harbours polyoma large T antigen, a high frequency of stable secondary transfection results. This process is referred to as supertransfection. Supertransfected plasmids can be maintained episomally during long-term culture and during differentiation in vitro. Expression of a β-galactosidase reporter from an episomal vector is both ubiquitous and stable, in contrast to the variegated and unstable expression usually observed after cDNA integration into the ES cell genome. Moreover, in the absence of integration, promoter strength is predictable and a range of expression levels can reliably be achieved by using different elements. We also show that episomal vectors can be used for efficient expression of both cytosolic and secreted proteins. These features should make this system invaluable for functional analyses of defined cDNAs and for direct expression screening of cDNA pools or libraries in ES cells.

TABLE 1
Comparison of β-galactosidase activities directed by various promoters in
transient and stable supertransfectants.

Relative β-gal
activity

| Promoter | transient | stable |
|---|---|---|
| SV40 e/p | 1.0 | 1.0 |
| hβAp | 1.1 | 0.7 |
| mPGKp | 0.5 | 0.5 |
| TKp | 0.1 | 0.1 |
| CAG | 19.0 | 1.8 |

5 × 10⁶ MG1.19 ES cells were supertransfected with 20 μg of vector DNAs. After 3 days culture for transient expression assay or 8 days selection with hygromycin B for stable expression assay, the β-galactosidase activity generated by these constructs was measured by ONPG assay. Results are normalised relative to activity generated by the SV40e/p construct. See ‘Materials and methods’ for construction details of vectors.

TABLE 2
Supertransfection of LIF and IL-2 expression vectors into MG1.19
ES cells.

| Vector | LIF in medium | No. of hyg^{r} stem cell colonies |
|---|---|---|
| pHPCAG-lif | + | 42,000 |
| pHPCAG-lif | − | 38,000 |
| pHPCAG-il2 | + | 48,000 |
| pHPCAG-il2 | − | 0 |

5 × 10⁶ MG1.19 ES cells were supertransfected with 20 μg of vector DNAs. After 8 days selection with 80 μg/ml of hygromycin B in the presence or absence of LIF, the number of stem cell colonies were scored.

TABLE 3
Efficiency of supertransfection of vectors with various selection markers.

| Selection marker | Drug for selection (μg/ml) | No. of resistant colonies |
|---|---|---|
| PGKhphpA | hygromycin B (80) | 50,000 |
| SVbsrpA | blasticidin S (4) | 12,600 |
| hCMVzeopA | zeocin (20) | 20,600 |

5 × 10⁶ MG1.19 ES cells were supertransfected with 20 μg of vector DNAs of episomal vectors, pBPCAG and pZPCAG, which carry bsr and zeo resistance genes respectively. After 8 days selection with the appropriate drug, the number of drug-resistant stem cell colonies were scored.

TABLE 4
Effects of overexpression of transgenes in ES cells using pHPCAG

| | Relative number of | Colony Size and |
|---|---|---|
| CDNA | hygro^{R} colonies | Morphology |
| None | 1.00 | Normal |
| lacZ | 0.64 | small |
| DIA/LIF | 0.87 | slightly small |
| IL-2 | 0.92 | slightly small |
| Rex-1 | 0.88 | Normal |
| Fgf-2 | 0.65 | Normal |
| Fgf-4 | 0.82 | Normal |
| Fgf-5 | 0.41 | Normal |
| Oct-1 | 0.17 | small |
| Oct-2 | 0.65 | slightly small |
| Oct-3/4 | 0.61 | differentiated |
| Oct-6 | 0.03 | some differentiation |
| c-jun | 0.47 | small |
| E1A | 0.08 | differentiated |
| Jak2 K/E | 0.75 | Normal |
| bcl-2 | 0.28 | small, spindle morphology |
| MAPKP | 1.38 | Normal |
| RXRα | 0.20 | some differentiation |
| RXRβ | 0.63 | Normal |
| RXRγ | 0.91 | Normal |
| COUP-T1 | 0.40 | some differentiation |
| HNF-4 | 0.05 | Normal |
| Stat1 | 0.10 | small |
| Stat3 | 0.52 | Normal |
| Stat4 | 0.16 | Normal |
| Stat3DON* | 0.14 | differentiated |

5 × 10⁶ ESMG1.19 cells were supertransfected with 20 μg of expression vectors and selected with 80 μg/ml of hygromycin B for 8 days. The numbers of drug-resistant colonies were counted and normalised relative to numbers obtained with empty vector.
*Stat3DON is the dominant interfering mutant form of Stat3 described by Akira et al. (1996).

1. A method of selecting a cell or a cell population, comprising:
(a) (i) transfecting a population of cells with a first vector that expresses a replication factor; or
(ii) otherwise obtaining a population of cells that express or will express the replication factor;
(b) transfecting cells in the population of cells with a second vector, wherein
(i) the second vector contains a first DNA in operative combination with a promoter for expression of the first DNA; and
(ii) extrachromosomal replication of the second vector is dependent upon presence within the cell of the replication factor;
(c) isolating a cell or cells that express the first DNA to form a first isolated cell or first isolated population of cells;
(d) culturing the first isolated cell or first isolated population of cells;
(e) subsequently isolating, from the culture of (d), a cell or cells that do not express the first DNA to form a second isolated cell or second isolated population of cells; and
(f) selecting the cell or cell population from the second isolated cell or second isolated population of cells.

2. The method of claim 1, wherein step (d) comprises culturing until the second vector is no longer episomally maintained in cells of the first isolated population.

3. The method of claim 1, wherein the first DNA encodes a selectable marker and step (d) comprises culturing in the absence of selection for the selectable marker until the second vector is no longer episomally maintained in cells of the first isolated population.

4. The method of claim 1, wherein step (c) comprises selecting for cells that express the first DNA.

5. The method of claim 1, wherein the first DNA encodes a selectable marker

6. The method of claim 5, wherein the selectable marker protects the cells from the effect of a cytotoxic agent added to cell culture medium.

7. The method of claim 6, wherein step (c) comprises isolating cells that express the marker by adding the cytotoxic agent to culture medium and retaining cells that survive.

8. The method of claim 6, wherein the selectable marker is a cell surface antigen.

9. The method of claim 8, wherein step (c) comprises isolating cells that express the marker using an antibody that binds to the cell surface antigen.

10. The method of claim 9, wherein step (e) comprises using an antibody that binds to the cell surface antigen to remove cells that express the marker, thereby isolating the cells that don't express the marker.

11. The method of claim 1, wherein the second vector contains the first DNA that encodes a first marker and also contains a second DNA that encodes a second marker and wherein the method comprises, in step (c) selecting for cells that express the first DNA and in step (e) selecting for cells that don't express the first DNA by selecting for cells that don't express the second DNA.

12. The method of claim 1, comprising isolating cells that don't express the first DNA by
picking an individual cell from the culture of (d);
growing the cell into a clonal population;
dividing the clonal population into first and second sub-populations;
transfecting the first sub-population with the second vector but not transfecting the second sub-population with the second vector; and
isolating the cells provided that cells of the first sub-population express the marker and that no cells of the second sub-population express the marker.

13. The method of claim 12, comprising isolating cells that don't express the first DNA by
picking an individual cell from the culture of (d);
growing the cell into a clonal population;
dividing the clonal population into first and second sub-populations;
transfecting the first sub-population with the second vector wherein the first DNA encodes resistance to a cytotoxic agent but not transfecting the second sub-population with the second vector;
isolating the cells provided that cells of the first sub-population are resistant to the cytotoxic agent and that no cells of the second sub-population are resistant to the cytotoxic agent.

14. The method of claim 1, wherein the replication factor is a viral replication factor.

15. The method of claim 1, wherein the cell is selected from the group consisting of a mammalian cell, and an avian cell.

16. The method of claim 15, where the cell is a pluripotent cell.

17. A method of selecting a mouse pluripotent cell, comprising:
(a) transfecting mouse pluripotent cells with a first vector that expresses a viral replication factor;
(b) transfecting the mouse pluripotent cells of (a) with a second vector, wherein
(i) the second vector contains a first DNA coding for a selectable marker in operative combination with a promoter for expression of the first DNA; and
(ii) the viral replication factor of step (a) replicates the second vector episomally;
(c) isolating the mouse pluripotent cells of step (b);
(d) culturing the mouse pluripotent cells of (c) until the second vector is no longer episomally maintained in the mouse pluripotent cells;
(e) isolating, from the culture of (d), a mouse pluripotent cell that does not express the first DNA; and
(f) selecting the isolated mouse pluripotent cell of step (e).

18. The method of claim 17, wherein the mouse pluripotent cell is an ES cell.

19. A method of obtaining a cell, comprising:—
(1) obtaining a cell which expresses a replication factor;
(2) transfecting the cell with a vector which requires the presence of the replication factor to be maintained episomally within the cell;
(3) expanding the cell into a plurality of cells; and
(4) selecting for a cell in the plurality of cells (i) which maintains the vector episomally, and (ii) in which the vector has not integrated into chromosomal DNA of the cell.

20. The method of claim 19, wherein the obtaining of (1) comprises transfecting the cell with a first vector that expresses a replication factor.

21. The method of claim 19, wherein the transfecting of (2) comprises transfecting the cells with a second vector that contains a first DNA encoding a selectable marker.

22. The method of claim 19, wherein the selecting of (4) comprises isolating cells that express the first DNA by isolating cells which have made the marker, culturing those isolated cells until the cells no longer maintain the vector episomally and then selecting for cells which do not express the first DNA.

23. The method of claim 19, wherein the selecting of (4) comprises isolating cells in which the vector is not integrated and then isolating cells which maintain the vector episomally.

24. The method of claim 19, comprising selecting the cells according to step (4) and then repeating steps (2) to (4) with that selected cell as the cell which is transfected in step (2).

25. A cell line obtained by the method of claim 1.

26. A cell line obtained by the method of claim 17.

27. A cell line obtained by the method of claim 19.

28. A vector comprising (a) an origin of replication which is bound by a viral replication factor, (b) DNA encoding a first selectable marker operatively linked to a first promoter and (c) DNA encoding a second selectable marker operatively linked to a second promoter, wherein the first selectable marker is resistance to a cytotoxic agent and the second selectable marker is a cell surface antigen, and wherein the vector does not include DNA encoding a replication factor that binds to the origin of replication

29. The vector of claim 28, wherein the second selectable marker is a flourescent protein.

30. A method of expressing a product DNA in a cell, comprising:
(a) (i) transfecting a population of cells with a first vector that expresses a replication factor; or
(ii) otherwise obtaining a population of cells that express or will express the replication factor;
(b) transfecting cells in the population of cells with a second vector, wherein
(i) the second vector contains a first DNA in operative combination with a promoter for expression of the first DNA; and
(ii) extrachromosomal replication of the second vector is dependent upon presence within the cell of the replication factor;
(c) isolating cells that express the first DNA to form a first isolated population of cells;
(d) culturing the first isolated population of cells;
(e) isolating, from the culture of (d), cells that do not express the first DNA to form a second isolated population of cells; and
(f) expressing the product DNA in a cell of the second isolated population of cells.

31. The method of claim 30, comprising transfecting a cell of the second isolated population of cells with a third vector containing the product DNA operatively linked to a promoter for expression of the product DNA.
